(19) United States
(12) Patent Application Publication
(10) Pub. No.: US 2015/0245612 A1
(43) Pub. Date: Sep. 3, 2015

(54) ACTIVE INGREDIENT FOR CONTROLLING SPIDER MITES

(71) Applicant: BAYER CROPSCIENCE AG, Monheim (DE)

(72) Inventors: Wolfgang THIELERT, Odenthal (DE); Holger WECKWERT, Leichlingen (DE); Leonardo PITTA, Leverkusen (DE); Michael MAUE, Langenfeld (DE); Heiko RIECK, Burscheid (DE)

(21) Appl. No.: 14/635,276

(22) Filed: Mar. 2, 2015

(30) Foreign Application Priority Data
Mar. 3, 2014 (EP) 14157445.9

(51) Int. Cl. A01N 43/56; C07D 231/14

(52) U.S. Cl. CPC A01N 43/56; C07D 231/14

(57) ABSTRACT

The present invention relates to novel uses of the compound of the formula (I) for controlling pests/spider mites from the order of Acari.

### CROSS REFERENCE TO RELATED APPLICATIONS

This application claims priority from EP 14157445.9 filed Mar. 3, 2014, the content of which is incorporated herein by reference in its entirety.

### BACKGROUND

1. Field of the Invention

The present application relates to the use of a known active ingredient for controlling spider mites (Tetranychidae family) in particular crops.

### DESCRIPTION OF RELATED ART

2. The compound of the formula (I)

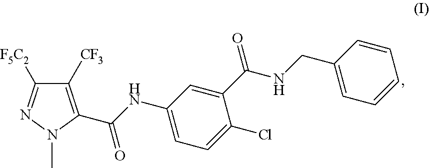

the preparation thereof and structurally similar compounds are known from WO 2010/051926. Further uses of this compound are also disclosed in WO 2010/051926.

Since the control of true spider mites in crops, especially field crops, is becoming increasingly important and the number of suitable active ingredients is falling, particularly because of regulatory demands in respect of ecotoxicological properties, the demand for suitable active ingredients is particularly high.

### SUMMARY

It has now been found that, surprisingly, the compound of the formula (I) is of particularly good suitability for controlling spider mites (Tetranychidae, order of Acari), especially for controlling spider mites of the Tetranychus urticae, Panonychus citri, Brevipalpus phoenicis, Phyllocoptruta oleivora, Aculops pelekassi, and Polyphagotarsonemus latus species on citrus plants. In addition, the compound of the formula (I) also has favourable ecotoxicological properties.

The aspect of the present invention relates to the use of the compound of the formula (I)

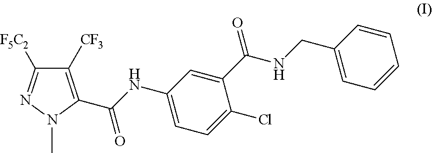

for controlling pests/spider mites from the order of Acari on citrus plants/in a citrus crop.

### DETAILED DESCRIPTION OF A PREFERRED EMBODIMENT

One embodiment relates to the above use, characterized in that the spider mite is selected from Tetranychus urticae, Panonychus citri, Brevipalpus phoenicis, Phyllocoptruta oleivora, Aculops pelekassi, and Polyphagotarsonemus latus.

One embodiment relates to the above use, characterized in that the spider mite is Panonychus citri.

One embodiment relates to the above use, characterized in that the spider mite is Brevipalpus phoenicis.

One embodiment relates to the above use, characterized in that the spider mite is Phyllocoptruta oleivora.

One embodiment relates to the above use, characterized in that the spider mite is Aculops pelekassi.

One embodiment relates to the above use, characterized in that the spider mite is Polyphagotarsonemus latus.

One embodiment relates to the above use, characterized in that the spider mite is Tetranychus urticae.

One embodiment relates to the above use, characterized in that the crop is selected from a group consisting of oranges (C. sinensis), lemons (C. limon), grapefruit (C. paradisi), limes (C. aurantifolia), mandarins (C. reticulata), clementines (C. clementina), satsumas (C. unshiu), bitter oranges (Citrus×aurantium L.), bergamot oranges (C. bergamia), pamplemousses (C. maxima), and kumquats (Fortunella genus).

One embodiment relates to the above use, characterized in that the crop is selected from a group consisting of oranges (C. sinensis), lemons (C. limon), grapefruit (C. paradisi), limes (C. aurantifolia), mandarins (C. reticulata), satsumas (Citrus unshiu) and clementines (C. clementina).

One embodiment relates to the above use, characterized in that the crop is selected from a group consisting of oranges (C. sinensis), lemons (C. limon), and satsumas (Citrus unshiu).

A further embodiment relates to the above uses, characterized in that the compound of the formula (I) is present in a mixture with at least one further insecticidal or acaricidal or nematicidal active ingredient.

A further embodiment relates to the above uses, characterized in that the compound of the formula (I) is present in a mixture with at least one further fungicidal active ingredient.

### DEFINITIONS

The terms “citrus”, “citrus crop” and “citrus plant” (Citrus) in the context of the present application are mutually interchangeable and relate to one or more plants from a genus of flowering plants in the Rutaceae family. The citrus plants include especially oranges (C. sinensis), lemons (C. limon), grapefruit (C. paradisi) and limes (various, usually C. aurantifolia, the key lime), mandarins (C. reticulata), clementines (C. clementina), satsumas (C. unshiu), bitter oranges (Citrus×aurantium L.), bergamot oranges (C. bergamia), pamplemousses (C. maxima), pomelos (e.g. Citrus×aurantium Pomelo group) and kumquats (Fortunella genus, for example Fortunella japonica (Syn. Citrus japonica)).

“Crop” in the context of the present application relates to a crop of plants. A crop of plants consists of at least one plant (or plant parts such as fruits) of one of the genera or species specified in this application. A crop in the context of the present application may thus relate to at least one wild plant (or plant parts such as fruits) of one of the genera or species specified in this application; or may preferably relate to at least one crop plant (or plant parts such as fruits), i.e. a plant which has been grown deliberately as a useful or ornamental plant by human intervention and/or processed for breeding purposes. A crop in the context of the present application may be part of a monoculture or part of a mixed culture. A monoculture is a term for the regional concentration of agricultural market production on one product (e.g. green beans, apples, pears, almonds, maize, cotton, soya beans, tomatoes, peppers or grapes). Usually, the person skilled in the art understands a monoculture to mean a crop rotation of useful plants consisting only of one useful plant species. A mixed culture may consist of wild plants of various species/genera, but in accordance with the invention encompasses one crop in the context of the present application, or a mixed culture may consist of crop plants of various species/genera, but in accordance with the invention encompasses one crop in the context of the present application. A citrus crop may thus consist of a citrus plant of a particular genus/species or of several citrus plants of the same genus/species.

All the plants and crops mentioned may also be transgenic plants or crops which have originated from the respective plants or crops through genetic modification.

The terms “compound of the formula (I)” and “compound (I)” are interchangeable.

The compound of the formula (I) is of particularly good suitability for use in the control of spider mites from the order of Acari in a citrus crop.

A preferred embodiment is directed to the inventive use of the compound of the formula (I) in the control of Tetranychus urticae, Panonychus citri, Brevipalpus phoenicis, Phyllocoptruta oleivora, Aculops pelekassi, and Polyphagotarsonemus latus.

A further preferred embodiment is directed to the inventive use of the compound of the formula (I) in the control of Panonychus citri, Brevipalpus phoenicis, Phyllocoptruta oleivora, Aculops pelekassi, and Polyphagotarsonemus latus.

A further preferred embodiment is directed to the use of the compound of the formula (I) in the control of spider mites/pests selected from Tetranychus urticae, Panonychus citri, Brevipalpus phoenicis, Phyllocoptruta oleivora, Aculops pelekassi, and Polyphagotarsonemus latus in a citrus crop selected from a group consisting of oranges (C. sinensis), lemons (C. limon), grapefruit (C. paradisi), limes (C. aurantifolia), mandarins (C. reticulata), clementines (C. clementina), satsumas (C. unshiu), bitter oranges (Citrus×aurantium L.), bergamot oranges (C. bergamia), pamplemousses (C. maxima), and kumquats (Fortunella genus).

A further preferred embodiment is directed to the use of the compound of the formula (I) in the control of spider mites/pests selected from Tetranychus urticae, Panonychus citri, Brevipalpus phoenicis, Phyllocoptruta oleivora, Aculops pelekassi, and Polyphagotarsonemus latus in a citrus crop selected from a group consisting of oranges (C. sinensis), lemons (C. limon), grapefruit (C. paradisi), limes (C. aurantifolia), mandarins (C. reticulata), satsumas (Citrus unshiu) and clementines (C. clementina).

A further preferred embodiment is directed to the use of the compound of the formula (I) in the control of spider mites/pests selected from Tetranychus urticae, Panonychus citri, Brevipalpus phoenicis, Phyllocoptruta oleivora, Aculops pelekassi, and Polyphagotarsonemus latus in a citrus crop selected from a group consisting of oranges (C. sinensis), lemons (C. limon), satsumas (Citrus unshiu), and limes (C. aurantifolia), more preferably oranges (C. sinensis), satsumas (Citrus unshiu) and lemons (C. limon).

A further preferred embodiment is directed to the use of the compound of the formula (I) in the control of spider mites/pests selected from Panonychus citri, Brevipalpus phoenicis, Phyllocoptruta oleivora, Aculops pelekassi, and Polyphagotarsonemus latus in a citrus crop selected from a group consisting of oranges (C. sinensis), lemons (C. limon), grapefruit (C. paradisi), limes (C. aurantifolia), mandarins (C. reticulata), clementines (C. clementina), satsumas (C. unshiu), bitter oranges (Citrus×aurantium L.), bergamot oranges (C. bergamia), pamplemousses (C. maxima), and kumquats (Fortunella genus).

A further preferred embodiment is directed to the use of the compound of the formula (I) in the control of spider mites/pests selected from Panonychus citri, Brevipalpus phoenicis, Phyllocoptruta oleivora, Aculops pelekassi, and Polyphagotarsonemus latus in a citrus crop selected from a group consisting of oranges (C. sinensis), lemons (C. limon), grapefruit (C. paradisi), limes (C. aurantifolia), mandarins (C. reticulata), satsumas (Citrus unshiu) and clementines (C. clementina).

A further preferred embodiment is directed to the use of the compound of the formula (I) in the control of spider mites/pests selected from Panonychus citri, Brevipalpus phoenicis, Phyllocoptruta oleivora, Aculops pelekassi, and Polyphagotarsonemus latus in a citrus crop selected from a group consisting of oranges (C. sinensis), lemons (C. limon), and limes (C. aurantifolia), more preferably from oranges (C. sinensis), satsumas (Citrus unshiu) and lemons (C. limon).

The following combinations of pest/spider mites and crop are particularly preferred embodiments of the inventive use of the compound of the formula (I) for controlling spider mites in particular crops:

Tetranychus urticae

Tetranychus urticae in oranges (C. sinensis),

Tetranychus urticae in lemons (C. limon),

Tetranychus urticae in grapefruit (C. paradisi),

Tetranychus urticae in limes (C. aurantifolia),

Tetranychus urticae in mandarins (C. reticulata),

Tetranychus urticae in clementines (C. clementina),

Tetranychus urticae in satsumas (Citrus unshiu).

Especially preferred is the inventive control of Tetranychus urticae in oranges (C. sinensis), satsumas (Citrus unshiu) and lemons (C. limon).

Panonychus citri

Panonychus citri in oranges (C. sinensis),

Panonychus citri in lemons (C. limon),

Panonychus citri in grapefruit (C. paradisi),

Panonychus citri in limes (C. aurantifolia),

Panonychus citri in mandarins (C. reticulata),

Panonychus citri in clementines (C. clementina),

Panonychus citri in satsumas (Citrus unshiu).

Especially preferred is the inventive control of Panonychus citri in oranges (C. sinensis), satsumas (Citrus unshiu) and lemons (C. limon). Brevipalpus phoenicis

Brevipalpus phoenicis in oranges (C. sinensis),

Brevipalpus phoenicis in lemons (C. limon),

Brevipalpus phoenicis in grapefruit (C. paradisi),

Brevipalpus phoenicis in limes (C. aurantifolia),

Brevipalpus phoenicis in mandarins (C. reticulata),

Brevipalpus phoenicis in clementines (C. clementina),

Brevipalpus phoenicis in satsumas (Citrus unshiu).

Especially preferred is the inventive control of Brevipalpus phoenicis in oranges (C. sinensis), satsumas (Citrus unshiu) and lemons (C. limon). Phyllocoptruta oleivora,

Phyllocoptruta oleivora in oranges (C. sinensis),

Phyllocoptruta oleivora in lemons (C. limon),

Phyllocoptruta oleivora in grapefruit (C. paradisi),

Phyllocoptruta oleivora in limes (C. aurantifolia),

Phyllocoptruta oleivora in mandarins (C. reticulata),

Phyllocoptruta oleivora in clementines (C. clementina),

Phyllocoptruta oleivora in satsumas (Citrus unshiu).

Especially preferred is the inventive control of Phyllocoptruta oleivora in oranges (C. sinensis), satsumas (Citrus unshiu) and lemons (C. limon).

Aculops pelekassi

Aculops pelekassi in oranges (C. sinensis),

Aculops pelekassi in lemons (C. limon),

Aculops pelekassi in grapefruit (C. paradisi),

Aculops pelekassi in limes (C. aurantifolia),

Aculops pelekassi in mandarins (C. reticulata),

Aculops pelekassi in clementines (C. clementina),

Aculops pelekassi in satsumas (Citrus unshiu).

Especially preferred is the inventive control of Aculops pelekassi in oranges (C. sinensis), satsumas (Citrus unshiu), grapefruit (C. paradisi) and lemons (C. limon).

Polyphagotarsonemus latus

Polyphagotarsonemus latus in oranges (C. sinensis),

Polyphagotarsonemus latus in lemons (C. limon),

Polyphagotarsonemus latus in grapefruit (C. paradisi),

Polyphagotarsonemus latus in limes (C. aurantifolia),

Polyphagotarsonemus latus in mandarins (C. reticulata),

Polyphagotarsonemus latus in clementines (C. clementina),

Polyphagotarsonemus latus in satsumas (Citrus unshiu).

Especially preferred is the inventive control of Polyphagotarsonemus latus in oranges (C. sinensis), satsumas (Citrus unshiu) and lemons (C. limon).

### Formulations

The present invention further relates to the inventive use of the compound of the formula (I) in the form of a formulation. The compound of the formula (I) can be used in various use forms for the inventive use, for example drench, drip and spray liquors, comprising the compound of the formula (I). In some cases, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further active agrochemical ingredients.

These are preferably formulations or use forms which comprise auxiliaries, for example extenders, solvents, spontaneity promoters, carriers, emulsifiers, dispersants, frost protectants, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are produced in a known manner, for example by mixing the compound of the formula (I) with auxiliaries, for example extenders, solvents and/or solid carriers and/or other auxiliaries, for example surfactants. The formulations are produced either in suitable facilities or else before or during application.

Auxiliaries used may be substances suitable for imparting special properties, such as certain physical, technical and/or biological properties, to the formulation of the compound of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, such as xylene, toluene or alkylnaphthalenes, chlorinated aromatic or aliphatic hydrocarbons, such as chlorobenzene, chloroethylene or methylene chloride, aliphatic hydrocarbons, such as cyclohexane, paraffins, mineral oil fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, and also water.

In principle, it is possible to use all suitable carriers. Useful carriers especially include, for example, ammonium salts and natural, finely ground rocks, such as kaolins, aluminas, talc, chalk, quartz, attapulgite, montmorillonite or diatomaceous earth, and synthetic, finely ground rocks, such as highly disperse silica, aluminium oxide and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, corn cobs and tobacco stalks.

Liquefied gaseous extenders or solvents can also be used. Especially suitable are those extenders or carriers which are gaseous at standard temperature and under standard pressure, for example aerosol propellants such as halohydrocarbons, or else butane, propane, nitrogen and carbon dioxide.

Examples of emulsifiers and/or foam formers, dispersants or wetting agents with ionic or nonionic properties, or mixtures of these surfactants, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds containing sulphates, sulphonates and phosphates, for example alkylaryl polyglycol ethers, alkyl sulphonates, alkyl sulphates, arylsulphonates, protein hydrolysates, lignosulphite waste liquors and methylcellulose. The presence of a surfactant is advantageous if one of the compounds of the formula (I) and/or one of the inert carriers is insoluble in water and when the application takes place in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethyl cellulose and natural and synthetic polymers in the form of powders, granules or latices, such as gum arabic, polyvinyl alcohol and polyvinyl acetate, or else natural phospholipids such as cephalins and lecithins and synthetic phospholipids. Further possible auxiliaries are mineral and vegetable oils.

Optionally, further auxiliaries may be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce the dynamic surface tension, for example dioctyl sulphosuccinate, or increase the viscoelasticity, for example hydroxypropylguar polymers.

Useful penetrants in the present context are all those substances which are typically used to improve the penetration of active agrochemical ingredients into plants. Penetrants are defined in this context by their ability to penetrate from the (generally aqueous) application liquor and/or from the spray coating into the cuticle of the plant and hence increase the mobility of active ingredients in the cuticle. The method described in the literature (Baur et al., 1997, Pesticide Science 51, 131-152) can be used for determining this property. Examples include alcohol alkoxylates, for example coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably contain between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (especially pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. Application is accomplished in a customary manner appropriate for the use forms.

### Mixtures

The compound of the formula (I) can also be used, on its own or in formulations thereof, in a mixture with one or more suitable fungicides, bactericides, acaricides, molluscicides, nematicides, insecticides, microbiologicals, beneficial organisms, herbicides, fertilizers, bird repellents, phytotonics, sterilants, synergists, safeners, semiochemicals and/or plant growth regulators, in order thus, for example, to broaden the spectrum of action, to prolong the duration of action, to increase the rate of action, to prevent repulsion or prevent evolution of resistance. In addition, active ingredient combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compound of the formula (I), on its own or in (commercial) formulations thereof and in the use forms prepared from these formulations, may be present in a mixture with further active ingredients or semiochemicals, such as attractants and/or bird repellents and/or plant activators and/or growth regulators and/or fertilizers and/or synergists. It is likewise possible to use the compound of the formula (I) and compositions thereof in mixtures with agents to improve plant properties, for example growth, yield and quality of the harvested material.

### Plants and Plant Parts

In the inventive use, it is possible to treat all plants and parts of plants with a compound of the formula (I). Plants are understood here to mean all plants and plant populations, such as desirable and undesirable wild plants or crop plants (including naturally occurring crop plants), for example maize, soya, tomatoes and other vegetables, cotton, and fruit plants (yielding apples, pears, and grapes or stone fruit, for example almonds). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Parts of plants shall be understood to mean all parts and organs of the plants above and below ground, such as shoot, leaf, flower and root, examples given being leaves, needles, stalks, stems, flowers, fruit bodies, fruits and seeds, and also tubers, roots and rhizomes. Parts of plants also include harvested material and vegetative and generative propagation material, for example cuttings, tubers, rhizomes, slips and seeds.

For the inventive use with the compound of the formula (I), the treatment of the plants and parts of plants with the compound of the formula (I) is effected directly or by allowing them to act on the surroundings, habitat or storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible in accordance with the invention to treat all plants and parts thereof. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been obtained by conventional breeding, by mutagenesis or by recombinant DNA techniques. They may be cultivars, varieties, biotypes or genotypes. The transgenic plants especially include Bt plants, which generate Bt toxins in the plants by virtue of the genetic material from Bacillus thuringiensis (for example by virtue of the CryIA(a), CryIA(b), CryIA(c), CryIIA, CryIIIA, CryIIIB2, Cry9c Cry2Ab, Cry3Bb and CryIF genes and combinations thereof). Preference is given to plants containing the CryIA(a), CryIA(b), CryIA(c) genes. The genes which impart the desired properties (“traits”) in question may also be present in combinations with one another in the transgenic plants.

The good efficacy of the compound of the formula (I) against pests from the family of the spider mites (Tetranychidae) in appropriate crops is apparent from the following examples:

### GABAmide—Spider Mite Field Trials

### Example 1

Panonychus citri test; outdoor spray treatment (METTCI)

To produce an appropriate active ingredient formulation, 1 part by weight of active ingredient is mixed with the stated amounts of water to the desired concentration. Added to the spray liquor is 0.1% a.i. rapeseed oil methyl ester.

Three two-year-old orange trees (Citrus sinensis) affected by all stages of the citrus red mite (Panonychus citri) are sprayed with an active ingredient formulation of each desired concentration. Application is effected with a motorized backpack sprayer. In accordance with common practice, the water application rate is 1000 l/Ha. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

B=treated plots
K=untreated control plots

In this test, the compound tested shows good efficacy compared to the spirodiclofen standard used:

| | | | | | Efficacy | |
|---|---|---|---|---|---|---|
| | | Animal | | Concentration | (% | |
| Substance | Structure | species | Plant | (g ai/ha/mch*) | Abbott) | Dat** |
| Spirodiclofen | 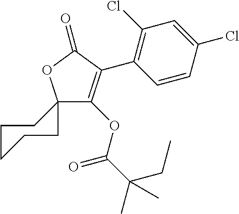 [SMILES: CCC(C)(C)C(=O)OC1=C(c2ccc(Cl)cc2Cl)C(=O)OC12CCCCC2] | METTCI | C. sinensis | 48 | 29 90 | 1 5 |
| Compound (I) (+ RME 0.1%) | 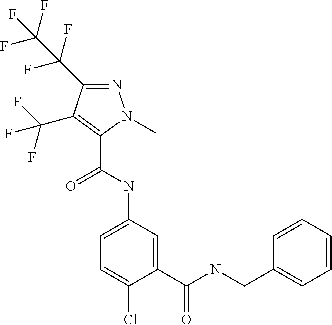 [SMILES: Cn1nc(C(F)(F)C(F)(F)F)c(C(F)(F)F)c1C(=O)Nc1ccc(Cl)c(C(=O)NCc2ccccc2)c1] | METTCI | C. sinensis | 50 100 | 85 99 99 100 | 1 5 1 5 |

*mch = metres crown height; **Dat = Days after treatment

### Example 2

### Panonychus citri Test; Outdoor Spray Treatment (METTCI)

To produce an appropriate active ingredient formulation, 1 part by weight of active ingredient is mixed with the stated amounts of water to the desired concentration. Added to the spray liquor is 0.1% a.i. Crovol.

Three two-year-old lemon trees (Citrus limon) affected by all stages of the citrus red mite (Panonychus citri) are sprayed with an active ingredient formulation of each desired concentration. Application is effected with a motorized backpack sprayer. In accordance with common practice, the water application rate is 1000 l/Ha. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

B=treated plots
K=untreated control plots

In this test, the compound tested shows good efficacy compared to the spirodiclofen standard used:

| | | | | | Efficacy | |
|---|---|---|---|---|---|---|
| | | Animal | | Concentration | (% | |
| Substance | Structure | species | Plant | (g ai/ha/mch*) | Abbott) | Dat** |
| Spirodiclofen | 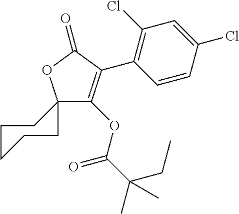 [SMILES: CCC(C)(C)C(=O)OC1=C(c2ccc(Cl)cc2Cl)C(=O)OC12CCCCC2] | METTCI | C. limon | 48 | 58 100 | 6 23 |
| Compound (I) (+ Crovol 0.1%) | 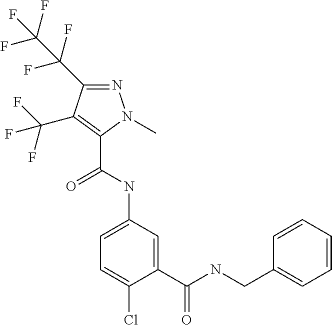 [SMILES: Cn1nc(C(F)(F)C(F)(F)F)c(C(F)(F)F)c1C(=O)Nc1ccc(Cl)c(C(=O)NCc2ccccc2)c1] | METTCI | C. limon | 100 | 90 99 | 6 23 |

*mch = metres crown height; **Dat = days after treatment

### Example 3

### Eutetranychus anneckei Test; Outdoor Spray Treatment (EUTEOR)

To produce an appropriate active ingredient formulation, 1 part by weight of active ingredient is mixed with the stated amounts of water to the desired concentration. Added to the spray liquor is 0.1% a.i. Crovol.

Three two-year-old orange trees (Citrus sinensis) affected by all stages of the oriental spider mite (Eutetranychus anneckei) are sprayed with an active ingredient formulation of each desired concentration. Application is effected with a motorized backpack sprayer. In accordance with common practice, the water application rate is 1000 l/Ha. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

B=treated plots
K=untreated control plots

In this test, the compound tested shows good efficacy compared to the cyenopyrafen standard used:

| | | | | | Efficacy | |
|---|---|---|---|---|---|---|
| | | Animal | | Concentration | (% | |
| Substance | Structure | species | Plant | (g ai/ha/mch*) | Abbott) | Dat** |
| Cyenopyrafen | 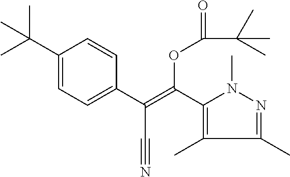 [SMILES: Cc1nn(C)c(/C(OC(=O)C(C)(C)C)=C(\C#N)c2ccc(C(C)(C)C)cc2)c1C] | EUTEOR | C. sinensis | 150 | 100 96 100 83 63 | 8 16 22 30 36 |
| Compound (I) (+ Crovol 0.1%) | 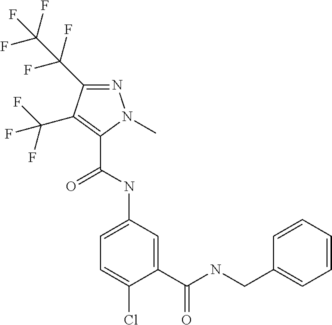 [SMILES: Cn1nc(C(F)(F)C(F)(F)F)c(C(F)(F)F)c1C(=O)Nc1ccc(Cl)c(C(=O)NCc2ccccc2)c1] | EUTEOR | C. sinensis | 100 | 99 99 100 98 93 | 8 16 22 30 36 |

*mch = metres crown height; **Dat = days after treatment

### Example 4

### Brevipalpus phoenicis Test; Outdoor Spray Treatment (BRVPPH)

To produce an appropriate active ingredient formulation, 1 part by weight of active ingredient is mixed with the stated amounts of water to the desired concentration. Added to the spray liquor is 0.1% a.i. Crovol.

Three eight-year-old orange trees (Citrus sinensis) affected by all stages of the false spider mite (Brevipalpus phoenicis) are sprayed with an active ingredient formulation of each desired concentration. Application is effected with a motorized backpack sprayer. In accordance with common practice, the water application rate is 1000 l/Ha. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

B=treated plots
K=untreated control plots

In this test, the compound tested shows good efficacy compared to the cyenopyrafen standard used:

| | | | | | Efficacy | |
|---|---|---|---|---|---|---|
| | | Animal | | Concentration | (% | |
| Substance | Structure | species | Plant | (g ai/ha/mch*) | Abbott) | Dat** |
| Cyenopyrafen | 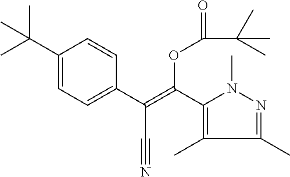 [SMILES: Cc1nn(C)c(/C(OC(=O)C(C)(C)C)=C(\C#N)c2ccc(C(C)(C)C)cc2)c1C] | BRVPPH | C. sinensis | 150 | 74.4 92.5 94.7 100 78 97.6 | 3 7 14 21 49 60 |
| Compound (I) (+ Crovol 0.1%) | 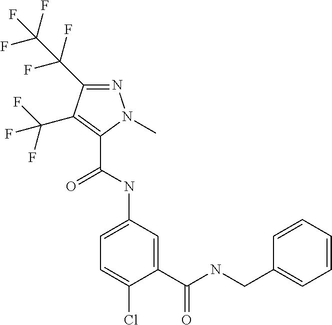 [SMILES: Cn1nc(C(F)(F)C(F)(F)F)c(C(F)(F)F)c1C(=O)Nc1ccc(Cl)c(C(=O)NCc2ccccc2)c1] | BRVPPH | C. sinensis | 100 | 88.4 95 100 100 92.7 100 | 3 7 14 21 49 60 |

*mch = metres crown height; **Dat = days after treatment

### Example 5

### Phyllocoptruta oleivora Test; Outdoor Spray Treatment (PHYUOL)

To produce an appropriate active ingredient formulation, 1 part by weight of active ingredient is mixed with the stated amounts of water to the desired concentration. Added to the spray liquor is 0.1% a.i. Crovol.

Three 15-year-old orange trees (Citrus sinensis) affected by all stages of the citrus rust mite (Phyllocoptruta oleivora) are sprayed with an active ingredient formulation of each desired concentration. Application is effected with a motorized backpack sprayer. In accordance with common practice, the water application rate is 1000 l/Ha. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

B=treated plots
K=untreated control plots

In this test, the compound tested shows good efficacy compared to the cyenopyrafen standard used:

| | | | | | Efficacy | |
|---|---|---|---|---|---|---|
| | | Animal | | Concentration | (% | |
| Substance | Structure | species | Plant | (g ai/ha/mch*) | Abbott) | Dat** |
| Cyenopyrafen | 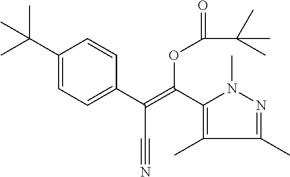 [SMILES: Cc1nn(C)c(/C(OC(=O)C(C)(C)C)=C(\C#N)c2ccc(C(C)(C)C)cc2)c1C] | PHYUOL | C. sinensis | 150 | 32 29 31 32 | 5 13 19 27 |
| Compound (I) (+ Crovol 0.1%) | 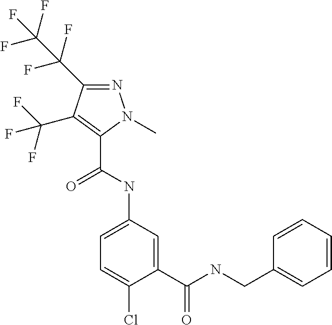 [SMILES: Cn1nc(C(F)(F)C(F)(F)F)c(C(F)(F)F)c1C(=O)Nc1ccc(Cl)c(C(=O)NCc2ccccc2)c1] | PHYUOL | C. sinensis | 75 100 | 36 45 64 72 77 84 94 96 | 5 13 19 27 5 13 19 27 |

*mch = metres crown height; **Dat = days after treatment

### Example 6

### Panonychus citri Test; Outdoor Spray Treatment (METTCI)

To produce an appropriate active ingredient formulation, 1 part by weight of active ingredient is mixed with the stated amounts of water to the desired concentration. Added to the spray liquor is 0.1% a.i. Crovol.

Three 5-year-old satsuma trees (Citrus unshiu) affected by all stages of the citrus red mite (Panonychus citri) are sprayed with an active ingredient formulation of each desired concentration. Application is effected with a motorized backpack sprayer. In accordance with common practice, the water application rate is 1850 l/Ha. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

B=treated plots
K=untreated control plots

In this test, the compound tested shows good efficacy compared to the spirodiclofen standard used:

| | | | | | Efficacy | |
|---|---|---|---|---|---|---|
| | | Animal | | Concentration | (% | |
| Substance | Structure | species | Plant | (g ai/ha/mch*) | Abbott) | Dat** |
| Spirodiclofen | 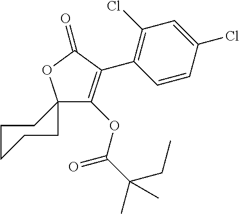 [SMILES: CCC(C)(C)C(=O)OC1=C(c2ccc(Cl)cc2Cl)C(=O)OC12CCCCC2] | METTCI | C. unshiu | 144 | 70.7 85.2 78 | 4 7 14 |
| Compound (I) (+ Crovol 0.1%) | 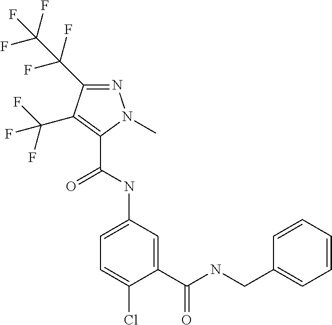 [SMILES: Cn1nc(C(F)(F)C(F)(F)F)c(C(F)(F)F)c1C(=O)Nc1ccc(Cl)c(C(=O)NCc2ccccc2)c1] | METTCI | C. unshiu | 100 | 100 98.1 97.6 | 4 7 14 |

*mch = metres crown height; **Dat = days after treatment

1. A compound of formula (I) 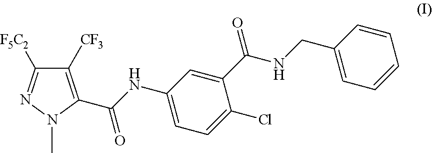
Capable of being used for controlling spider mites from the order of Acari in a citrus crop.

2. A compound according to claim 1, wherein the spider mite is selected from Tetranychus urticae, Panonychus citri, Brevipalpus phoenicis, Phyllocoptruta oleivora, Aculops pelekassi, and Polyphagotarsonemus latus.

3. Compound according to claim 1, wherein the spider mite is Panonychus citri.

4. Compound according to claim 1, wherein the spider mite is Brevipalpus phoenicis.

5. Compound according to claim 1, wherein the spider mite is Phyllocoptruta oleivora.

6. Compound according to claim 1, wherein the spider mite is Aculops pelekassi.

7. Compound according to claim 1, wherein the spider mite is Polyphagotarsonemus latus.

8. Compound according to claim 1, wherein the spider mite is Tetranychus urticae.

9. Compound according to claim 1, wherein the crop is selected from a group consisting of oranges (C. sinensis), lemons (C. limon), grapefruit (C. paradisi), limes (C. aurantifolia), mandarins (C. reticulata), clementines (C. clementina), satsumas (C. unshiu), bitter oranges (Citrus×aurantium L.), bergamot oranges (C. bergamia), pamplemousses (C. maxima), and kumquats (Fortunella genus).

10. Compound according to claim 1, wherein the crop is selected from a group consisting of oranges (C. sinensis), lemons (C. limon), grapefruit (C. paradisi), limes (C. aurantifolia), mandarins (C. reticulata), clementines (C. clementina) and satsumas (Citrus unshiu).

11. Compound according to claim 1, wherein the crop is selected from a group consisting of oranges (C. sinensis), lemons (C. limon), and satsumas (Citrus unshiu).
